(19) United States
(12) Patent Application Publication
(10) Pub. No.: US 2025/0076291 A1
(43) Pub. Date: Mar. 6, 2025

(54) BIOSELECTIVE SENSOR SURFACES

(71) Applicant: Southwest Research Institute, San Antonio, TX (US)

(72) Inventors: Steven T. WELLINGHOFF, San Antonio, TX (US); Carlos Martin CANTÚ, San Antonio, TX (US); Nicholas P. MCMAHON, San Antonio (TX)

(21) Appl. No.: 18/459,576

(22) Filed: Sep. 1, 2023

(51) Int. Cl. G01N 33/543; G01N 33/53; G01N 33/569

(52) U.S. Cl. CPC G01N 33/54353; G01N 33/5308; G01N 33/54373; G01N 33/56983; G01N 2333/11

(57) ABSTRACT

Functionalizing a sensor surface with a polydopamine or polynorepinephrine coating covalently bonded to a multi-arm poly(ethylene oxide) that is covalently bonded to a deglycosylated native avidin protein having available biotin binding sites for functionalization with biotinylated aptamers or proteins/peptides that can selectively bind to target biomolecules.

### FIELD

The present invention is directed at functionalizing a sensor surface with a polydopamine or polynorepinephrine coating covalently bonded to a multi-arm poly(ethylene oxide) that is covalently bonded to a deglycosylated native avidin protein (DGNAP) having biotin binding sites bonded to a bait aptamer. The sensor can be employed for detecting a biomolecule target such as a suspect/targeted virus.

### BACKGROUND

Biosensors have become an important tool in the field of diagnostics. Modification of the biosensor surface has been recognized as important to produce biomolecule detection platforms. Various avenues have therefore been pursued to provide a functionalized sensor surface with the desired result of immobilizing a target biomolecule.

In U.S. Pat. No. 6,638,728, there is reported new protein coated surfaces which are said to have a high capacity for capturing target molecules, thus yielding assays with enhanced sensitivity. The surface is described as containing a streptavidin, avidin or a deglycosylated native avidin protein coating in polymeric form, where polymerization is controlled to an extent such that the polymer is predominantly dimers, trimers and tetramers of the native molecules.

U.S. Pat. No. 8,580,760 reports on the development of an aptamer as a ligand molecule that is capable of specifically binding to a target substance. More specifically, a viral hemorrhagic septicemia virus (VHSV)-binding aptamer sequence having an activity of binding to at least one VHSV or polypeptide expressed by VHSV and suppressing VHSC infection of a host.

U.S. Publication No. 2020/0178857 reports on an aptamer based portable diagnostic medical device. More specifically, a sensor component within the device may include one or more aptamers that permit chemical binding of at least one biomarker of interest.

U.S. Pat. No. 9,272,075 reports on a substrate with a surface having a hydrophilic coating comprising cross-linked copolymers that is said to be covalently attached to the surface of the substrate. The coatings are said to be suitable for use on substrates such as medical devices, analytical devices, separation devices and other industrial articles. The patent also identifies that in one aspect, the invention provides a substrate having a first coating and a second coating, wherein the first coating is a surface priming coating of polydopamine and the second coating is a hydrophilic coating comprising certain hydrophilic monomers and hydrophilic polymers bearing one or more alkene or alkyne groups.

### SUMMARY

A method for functionalizing a sensor surface to selectively bind a target molecule comprising providing a sensor surface, coating the surface with either polydopamine or polynorepinephrine, and providing a multi-arm poly(ethylene oxide) having the following formulae:

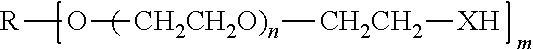

wherein n has a value of 10 to 45, m has a value of 2-12, R is an alkyl group providing carbon functionality to bond to the polyethylene oxide (PEO), X is S, —NH or O thereby providing available thiol, amine or hydroxy functionality. This is followed by covalently bonding a portion of the available thiol, amine or hydroxy functionality on the multi-arm poly(ethylene oxide) to the polydopamine or polynorepinephrine and providing a maleimide conjugated deglycosylated native avidin protein (DGNAP) having biotin binding sites, the maleimide conjugated deglycosylated native avidin protein having the following general structure:

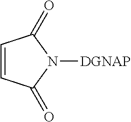

One then can covalently bond a portion of said available thiol, amine or hydroxy functionality on said multi-arm poly(ethylene oxide) to said maleimide conjugated deglycosylated native avidin protein having the biotin binding sites. In the particular case of a hydroxy functional group, it is contemplated that such reaction would be promoted with the aid of a catalyst. For example, silylation and a titanium-based catalyst. This may then be followed with binding of a biotinylated bait aptamer to the biotin binding sites where the biotinylated bait aptamer binds to a specific target molecule.

The present invention also includes a method of detecting a target virus comprising providing a sensor surface coated with polydopamine or polynorepinephrine, covalently bonded to a multi-arm poly(ethylene oxide) wherein the multi-arm poly(ethylene oxide) is covalently bonded to a maleimide conjugated deglycosylated native avidin protein having biotin binding sites, which binding sites are then bound to a biotinylated bait aptamer which binds to a target virus. This is then followed by exposing the sensor surface to a fluid sample containing a target virus and detecting the presence of the target virus.

The present invention also includes a sensor for detecting a target virus comprising a sensor surface coated with polydopamine or polynorepinephrine, covalently bonded to a multi-arm poly(ethylene oxide) wherein the multi-arm poly(ethylene oxide) is covalently bonded to a maleimide conjugated deglycosylated native avidin protein having biotin bonding sites which biotin binding sites are bonded to a biotinylated bait aptamer wherein the biotinylated bait aptamer binds to target virus.

### BRIEF DESCRIPTION OF THE DRAWINGS

FIG. 1 shows the binding of biotin-conjugated gold nanoparticles to deglycosylated native avidin protein (DGNAP)-maleimide/four arm HS-PEO/PNE and four arm HS-PEO/PNE coated Au sensors with QCM-I.

FIG. 2 shows the average frequency shift recorded on QCM-I after biotinylated gold nanoparticles were added to deglycosylated native avidin protein (DGNAP)-maleimide/four arm HS-PEO/PNE and four arm HS-PEO/PNE coated Au sensors.

FIG. 3A shows the frequency change after static H1N1 virus incubation on the identified sensor surfaces.

FIG. 3B shows the dissipation variation after static H1N1 virus incubation on the identified sensor surfaces.

FIG. 4A shows the frequency change after H1N1 virus incubation on the identified sensor surfaces under continuous convection conditions.

FIG. 4B shows the dissipation variation after H1N1 virus incubation on the identified sensor surfaces under continuous convection conditions.

### DETAILED DESCRIPTION

The present invention is directed at a method and apparatus that employs a coating composition that is suitable for functionalizing an acoustic, optical, or electrochemical sensor surface to selectively bind a target molecule or a target virus. For example, the target molecule can include a protein predominant on the surface of the H1N1 virus.

Preferably, the substrate sensor surface comprises a bioselective sensor surface, which is a reference to a surface that is capable of generating a signal proportional to the concentration of a particular chemical compound or series of compounds. The sensor herein may preferably include, e.g., a quartz crystal microbalance (QCM) sensor, typically employed to measure the interactions of molecules, polymers, and biological assemblies with a sensor surface, in air or liquid, label-free, and in real time. In this category of sensors, one may also utilize QCM-I, which refers to a quartz crystal microbalance with impedance spectroscope, or QCM-D, which is a quartz crystal microbalance with dissipation monitoring.

The target or suspect virus is in a fluid that contains the virus. The fluid sample can be liquid or gas. The fluid sample may therefore comprise ambient air, exhaled air, bodily fluids such as blood, saliva, mucus, urine, etc., aqueous solutions, liquids containing tissue samples, membrane samples, and others. The fluid samples may be used directly or diluted in a selected solution. The fluid can be applied to the coated sensor surface herein by any suitable method, such as dropping a liquid sample, flowing the liquid sample across the coated sensor surface, exposing the coated sensor surface to exhaled breath, etc.

The target or suspect virus herein includes any viral biomolecule with any chemical entity directly or indirectly related to the presence of viruses by indicating a current or previous infection with the virus. Non-limiting examples of biomolecules include whole viruses, antibodies, antigens, viral proteins, viral RNA, viral DNA, and viral biomarkers.

Initially, a substrate surface, such as a sensor surface, is coated with polydopamine (PDA) or polynorepinephrine (PNE). Such coating may preferably have a thickness of less than or equal to 100 nm, such as in the range of 0.1 nm to 100 nm, including all values and increments therein. It is contemplated that the sensor surface, before coating with PDA or PNE, may optionally include an initial parylene layer comprising substituted (e.g., halogen or chlorine substituted) para-benzenediyl rings (—C₆H₄—) connected by 1,2-ethanediyl bridges (—CH₂—CH₂—).

Polydopamine is reference to a polymer comprising catechol functionality along with sites, such as electron deficient alkene sites, suitable for Michael addition of, e.g., nucleophilic group such as a thiol group (—SH), which then provides covalent bond attachment. Polydopamine herein may therefore have the following structures A and B:

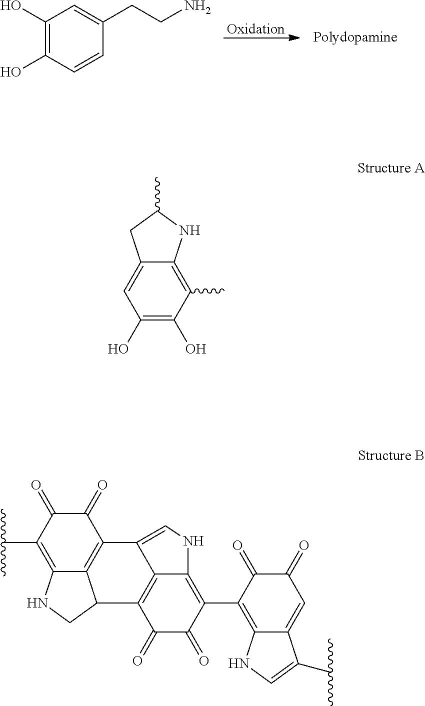

The polymerization of norepinephrine to structures A and B may be illustrated as follows:

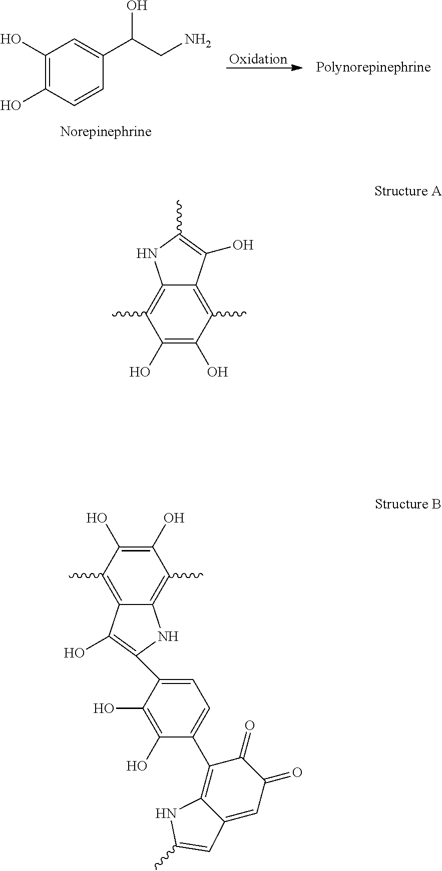

The sensor surface coated with either polydopamine or polynorepinephrine is then preferably treated with a multi-arm poly(ethylene oxide) having the following general structure,

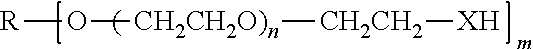

wherein R is reference to alkyl group providing carbon functionality to bond to the poly(ethylene oxide), e.g., a tetra-substituted carbon, that allows for the multi-arm structure, n is the number of repeating units of the ethylene oxide group, m is the number of arms present, and X may be S, —NH or O. Preferably X is S thereby providing thiol group functionality (—SH). The value of n is such that the molecular weight of the repeating ethylene oxide groups in the arm provides a molecular weight in the preferred range of about 500 to 2000. Accordingly, n preferably has a value in the range of 10 to 45. The value of m, or the number of arms present, is preferably in the range of 2-12, more preferably 2-8, 2-6, 3-6 and most preferably m has a value of 4. The molecular wight of the multi-arm poly(ethylene oxide) itself may preferably have a value in the range of 4000 to 8000, more preferably in the range of 4500 to 5500.

Accordingly, a particular preferred multi-arm poly(ethylene oxide) is a thiol-terminated polyalkylene oxide (where X is the formula below is S) and comprises the following 4-arm structure:

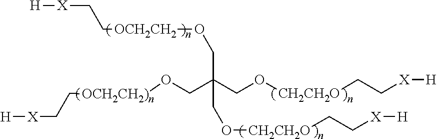

In the above, the value of n, which is the number of repeating units of the ethylene oxide unit, is again preferably set in the range of 10-45 so that the molecular weight of the poly(ethylene oxide) unit is in the range of about 500 to 2000.

The multi-arm poly(ethylene oxide) described above with either thiol (—SH), amine (—NH₂) and/or hydroxy (—OH) functionality, can then be reacted and covalently bonded to either the polydopamine (PDA) and/or polynorepinephrine (PNE) coating noted above, via a Michael addition reaction. A Michael addition reaction is a reference to the reaction of the aforementioned functional groups (—SH, —NH₂ and/or —OH) with a reactive alkene within the structure of the PDA or PNE coating. As noted above, in the case of an —OH group, it is contemplated that one would utilize a catalyst, for example, silylation and a titanium-based catalyst. In particular, a reaction of the aforementioned functional groups can occur with any activated double bond with the PDA or PNE with electron-withdrawing groups.

For example, in the case of PDA, when X is S, and a thiol end-group is present on the multi-arm polyethylene oxide, one then forms a covalently bonded PDA-PEO conjugate. In the structure below, only one covalent bond of the multi-arm PEO is shown to be connected to the PDA, and it can be appreciated that other arms may be similarly connected. However, it can be appreciated that the reaction may be adjusted so that at a given bonding location, there are remaining free thiol (—SH) groups, as shown. Accordingly, in the case of a 4-arm thiol terminated PEO, there can be up to 3 remaining free thiol (—SH) groups at any attachment location to the polydopamine coating.

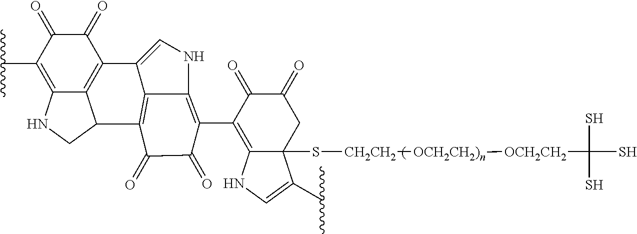

The above may be conveniently abbreviated as follows:

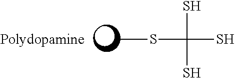

In the case of a polynorepinephrine (PNE) coating on a given substrate-sensor surface, the conjugation with the multi-arm PEO having thiol (—SH), amine (—NH₂) and/or hydroxy (—OH) end-group functionality can again preferably proceed by a Michael addition to an activated alkene in the PNE coating, by way of example, a Michael addition reaction of the thiol-terminated multi-arm PEO with an alkene bond in PNE. It is therefore contemplated that in the case of a 4-arm thiol terminated PEO, there can be up to 3 remaining free thiol (—SH) groups at an attachment location on the PNE coating:

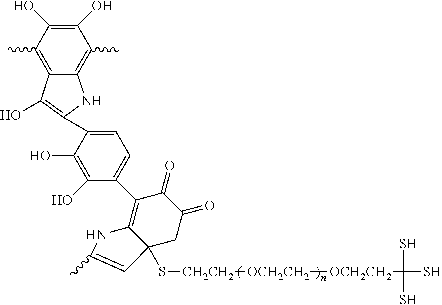

The above may be conveniently abbreviated as follows:

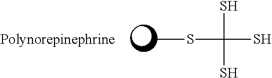

As may now be appreciated, the free thiol group (—SH) functionality and/or free amine (—NH₂) and/or hydroxy (—OH) groups remaining on the multi-arm PEO, covalently bonded to either PDA or PNE can now be selectively reacted with a maleimide conjugated deglycosylated native avidin protein having biotin binding sites. Deglycosylated native avidin protein (“DGNAP”) is a reference to a deglycosylated native avidin protein from egg whites, with a mass of approximately 60,000 daltons. It is commercially available from ThermoFisher under the name NeutrAvidin™ Protein. As a result of carbohydrate removal, lectin binding is reduced and as noted, biotin-binding affinity is retained. The maleimide conjugated deglycosylated native avidin protein (DGNAP) from egg whites deglycosylated native avidin protein can be indicated as follows:

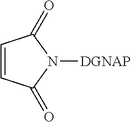

Reaction of the free thiol functional groups of the multi-arm PEO covalently attached herein to either PDA or PNP coating on a substrate surface, may be illustrated as follows. First, in the case of PDA:

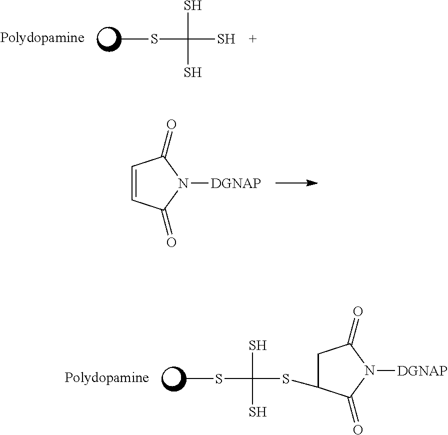

Second, in the case of PNE:

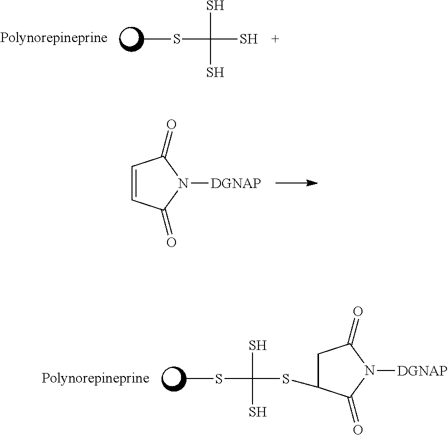

In connection with the above, it is noted that initially, to confirm efficacy, a commercial, end biotinylated single-strand “bait” aptamer, directed against the hemagglutinin (HA) “prey” protein, predominant in the surface of the H1N1 virus, was bound to the biotin binding sides on the deglycosylated native avidin protein attached to the maleimide functionality, which as noted above, is covalently attached to the multi-arm PEO, that is itself covalently attached to either a polydopamine or polynorepinephrine coating on a substrate (sensor) surface. The bonding of the HA prey protein to the aptamer functionalized deglycosylated native avidin protein was verified by QCM-I. The bonding of the live H1N1 protein to the aptamer functionalized surface was also verified by QCM-I. It should be noted that reference to a bait aptamer herein is reference to a nucleic acid binding species, including sequences of DNA, RNA, XNA or peptides that bind a specific target molecule such as a targeted virus having a prey protein and X is a non-natural nucleotide.

In the broader context of the present invention, the detection herein of the target or suspect virus on the coated sensor includes molecular recognition of any chemical compound or group that selectively binds with the target virus. Non-limiting examples of molecular recognition groups herein include aptamers, antigens, and antibodies.

### WORKING EXAMPLES

### Example 1: Conjugation of Deglycosylated Native Avidin Protein-Maleimide on Four Arm HS-PEO/PDA Surface

QCM-I gold sensors (14 mm, Au-coated quartz crystals, Gamry Instruments) were cleaned with ultrasonication in ethanol and ultrapure water, followed by gentle drying with N₂ gas before testing with QCM-I (Gamry Instruments). Frequency changes due to solvent effects and temperature, which can lead to background noise during QCM-I recordings, were accounted for during PDA and four arm HS-PEO conjugations. QCM-I recordings were done in buffer only to compare to PDA coating, as well as in buffer only to compare to four arm HS-PEO to PDA conjugation. The differences in frequency change were then tested for statistical significance and then deposited mass/cm² estimated.

PDA (2 mg/ml) was coated by depositing the solution on the Au electrodes in standard pH 8.5, 10 mM Tris buffer at 25° C. for 10 min. The PDA surface was functionalized by a Michael addition-based reaction with the 4 multi-arm PEO thiols. A pH 8.5, 5 mM TCEP ((tris(2-carboxyethyl)phosphine)-HCl, 10 mM EDTA (ethylenediamine tetra-acetic acid)/Tris (tris(hydroxymethyl) aminomethane) buffer was used for conjugation of 4 multi-armed HS-PEO (2.5 mg/ml, MW: 5000 Da, arm MW: 1250 Da, Creative PEG Works) to PDA for 2 hours at 50° C. See Table 1 below. Given that the mass deposited was higher than 92.433 ng/cm², which is the calculated deposited mass for a monolayer, the deposited four arm HS-PEO layer is present as a multi-layer. Since the thickness for a monolayer is calculated to be 1.569 nm, the thickness is estimated to be 19.610±7.181 nm.

TABLE 1
Summary Of QCM-1 Mass Measurements
For SH-PEO Conjugation to PDA

| Sensor | Bound Mass | Bound Molecules |
|---|---|---|
| Number | (ng/cm²) | (molecules/cm²) |
| 1 | 1736.5 | 2.1E+14 |
| 2 | 1436.5 | 1.7E+14 |
| 3 | 1404.5 | 1.7E+14 |
| 4 | 803.12 | 9.7E+13 |
| 5 | 814.10 | 9.8E+13 |
| Average | 1155.20 +/− 423.05 | 1.4E14 +/− 5.1E14 |

The remaining thiol groups were functionalized with maleimide activated deglycosylated native avidin protein (ThermoFisher Scientific) to bind it covalently to the surface. Conjugation of deglycosylated native avidin protein-maleimide (0.2 mg/ml) to four arm HS-PEO was done in a nitrogen purged pH 7.0-7.2, 10 mM EDTA, 0.1 M phosphate buffer at 25° C. for 2 hours. The four arm HS-PEO/PDA surface was reduced with pH 8.5, 5 mM TCEP-HCl, 10 mM EDTA Tris buffer prior to conjugation of deglycosylated native avidin protein-maleimide in phosphate buffer to eliminate any disulfide bonds which would hamper the deglycosylated native avidin protein-maleimide reaction.

### Example 2

### FTIR Characterization of PDA Films and PEO Conjugation

All PDA and four arm HS-PEO/PDA coated Au surfaces on QCM-I sensors were cleaned with ultrapure water sonication before being assessed with FTIR. A fresh uncoated sensor control, which was cleaned in ethanol and ultrapure water ultrasonication, was compared with PDA and four arm HS-PEO/PDA coated sensors. Single bounce attenuated total reflectance (ATR-FTIR) analysis was used to assess the functional groups found on PDA and four arm HS-PEO/PDA coatings. The presence of PDA and four arm HS-PEO/PDA coating was verified with these FTIR measurements when compared with a QCM sensor Au surface with no coating.

### Example 3

### Deglycosylated Native Avidin Protein Au Nanoparticle Conjugate Based Surface Coating On Quartz Crystal Gold Electrode

Conjugation of biotin functionalized Au nanoparticles will increase the surface area on sensor surfaces. By increasing the surface area and therefore presenting more interacting targeting ligands, the detection capabilities can be optimized. Biotinylated Au nanoparticles purchased from suppliers are also coated with PEO, which will reduce any possible non-specific binding.

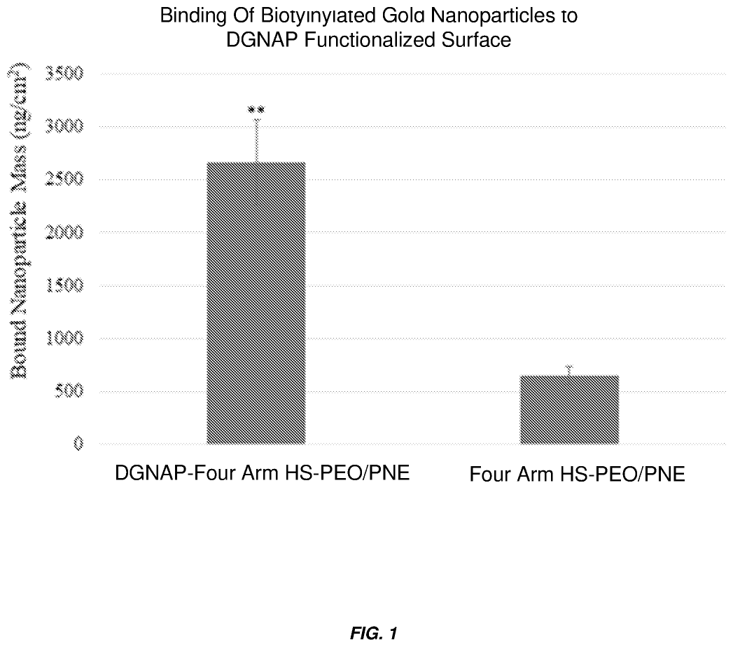

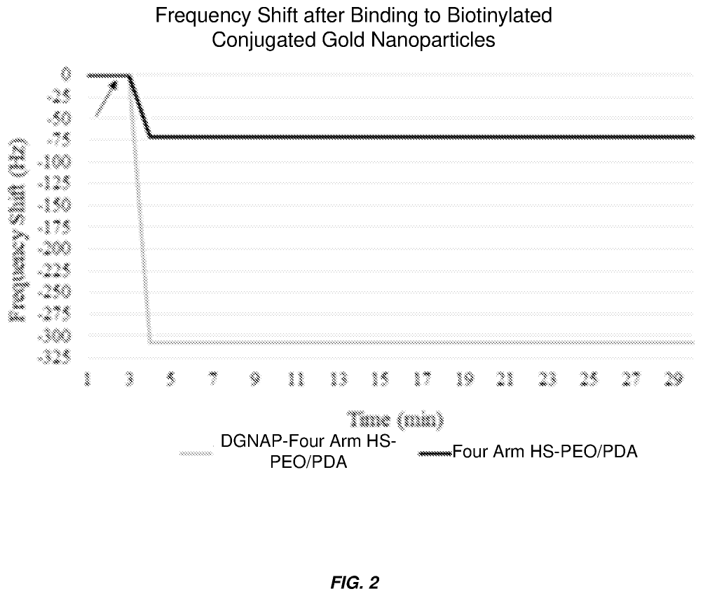

100 nm biotin conjugated Au nanoparticles (NNCrystal U.S. Corporation) were bound in pH 7.4, 0.1 M PBS at 4° C. to maleimidated deglycosylated native avidin protein that had been previously bound to four arm HS-PEO/PDA/Au surface through the reaction of the maleimide groups with a remaining free thiol. The conjugation of the biotinylated Au-nanoparticles to the free biotin binding sites of deglycosylated native avidin protein-maleimide was validated by QCM-I. See FIG. 1 which shows the binding of biotin-conjugated gold nanoparticles to deglycosylated native avidin protein functionalized and non-functionalized surfaces with QCM-I, three samples for each condition. Frequency shift data was employed to derive the mass bound using the Sauerbrey equation, p<0.01. See also FIG. 2, which shows the average frequency shift recorded on QCM-I after biotin gold nanoparticles were added to deglycosylated native avidin protein maleimide/four arm HS-PEO/PDA and four arm HS-PEO/PDA coated Au sensors. The black arrow in FIG. 2 denotes the end of the baseline measurements, which was followed by perfusion of a biotin-Au to flow cells, followed by deionized water washing of flow cells, and then drying with N₂ gas. Measurements were then resumed after N₂ drying to calculate bound mass, three samples for each condition. The results therefore show binding of a 100 nm analyte that is within the diameter size range of the H1N1 virus (80-120 nm range), albeit in the case of Au, aided by gravitational settling.

### Example 4: PNE Coating

A PNE coating was implemented in pH 8.5, 10 mM Tris at 60° C. for 3.5 hr with 2 mg/ml norepinephrine monomer. HS-PEO (4 arm-total molecular weight of 5000 Da, arm molecular weight of 1,250 Da-2.5 mg/ml) was then conjugated to PNE overnight in pH 8.5, 5 mM TCEP-HCl, 10 mM EDTA, Tris buffer at 50° C. Deglycosylated native avidin protein-maleimide (0.2 mg/ml) was then conjugated to the four arm HS-PEO layer in nitrogen purged pH 7.0-7.2, 10 mM EDTA, 0.1 M phosphate buffer at 25° C. for 2 hours. The PNE/four arm HS-PEO surface was reduced with pH 8.5, 5 mM TCEP-HCl, 10 mM EDTA Tris buffer prior to conjugation of deglycosylated native avidin protein-maleimide in phosphate buffer.

In addition, a biotinylated quantum dot (QD, 655 nm ThermoFisher) fluorescent experiment that involved use of a microplate reader was used to further confirm the conjugation of deglycosylated native avidin protein-maleimide. Thus, a biotinylated-QD reaction solution (ThermoFisher kit) was prepared with a concentration of 13 nM in dilution working buffer supplied with the kit. The functionalized experimental and control QCM-I sensors were incubated for 1 hour at room temperature in 0.5 ml of reaction solution. After incubation, the sensors were washed 3× each with the dilution working buffer and then resuspended in a 24 well plate with dilution working buffer to determine the amount of sensor surface bonded QD. A standard curve was prepared to calculate the surface density of bound biotinylated-QD on experimental and control sensors (Table 2).

TABLE 2
Biotinylated-QD Surface Density on QCM Sensors

| | Biotin-QD |
|---|---|
| | Surface Density |
| Surface Density Condition | (Molecules/cm²) |
| Total Biotin-QD Surface Density | (2.4 +/− 0.52) × 10¹¹ |
| Non-Specifically Bound Biotin-QD Density | (6.05 +/− 3.4) × 10¹⁰ |
| Specifically Bound Biotin-QD Density | (1.31 +/− 0.47) × 10¹¹ |

PNE deposition and then four arm HS-PEO conjugation on PNE (Table 3—dry film) was measured by QCM-I-the presence of which was also verified by FTIR. Subsequent deglycosylated native avidin protein-maleimide bonding to four arm HS-PEO/PNE was also followed with QCM-I, albeit without a buffer control to account for background noise (Table 4—dry film). The number of bound deglycosylated native avidin protein-maleimide molecules was on the order of 1/327 the number of four arm HS-PEO molecules bound to the PNE surface (Table 4—dry film).

TABLE 3
Bound HS-PEO To PNE Coated QCM Sensors

| | Bound Mass | Bound Molecules |
|---|---|---|
| Sensor # | (ng/cm²) | (molecules/cm²) |
| 1 | 1781 | 2.145 × 10¹⁴ |
| 2 | 2038 | 2.455 × 10¹⁴ |
| 3 | 2413 | 2.906 × 10¹⁴ |
| 4 | 2142 | 2.580 × 10¹⁴ |
| Average | 2094 +/− 261.5 | (2.522 +/− 0.3149) × 10¹⁴ |

TABLE 4
Bound Deglycosylated Native Avidin Protein -Maleimide
Based On QCM-1 Measurements

| Sample | Bound Mass | Bound Molecules |
|---|---|---|
| Number | (ng/cm²) | (molecules/cm²) |
| 1 | 687.2 | 6.998 × 10¹² |
| 2 | 853.8 | 8.569 × 10¹² |
| 3 | 914.9 | 9.183 × 10¹² |
| Average | 822.0 +/− 113.3 | (8.250 +/− 1.127) × 10¹² |

AFM was used to measure the PNE topology on the Au surface. The surface roughness of the PNE layer was significantly less than the PDA layer (Table5).

TABLE 5
Comparison Of PNE And PDA Roughness
Values Derived From AFM

| Roughness | | | |
|---|---|---|---|
| Value | Rq (nm) | Ra (nm) | Rmax (nm) |
| PNE Coated | 1.6 +/− 0.07** | 1.2 +/− 0.09* | 26.9 +/− 4.91 |
| Gold Sensor | | | |
| PDA Coated | 4.1 +/− 0.16 | 1.7 +/− 0.12 | 280.66 +/− 294.04 |
| Gold Sensor | | | |

(*p < 0.05, **p < 0.01, n = 3 for both conditions)

### Example 5—Conjugation of DNA Aptamer to Sensor Surface and Assessment of Its Detection Capability

Hemagglutinin (HA) and neuraminidase (NA) are the dominant glycoproteins found on the membrane of the H1N1 virus, in addition, there are on average 300-400 HA proteins versus 40-50 NA spikes on an average virion. Due to these characteristics the target for the aptamer was chosen to be the HA protein. Commercially available biotinylated DNA HA specific aptamer (Creative Biolabs) was chosen as the targeting ligand that will bind to the target protein hemagglutinin (HA) on H1N1 virus (H1N1, A/Puerto Rico/8/1934).

The deglycosylated native avidin protein-maleimide/four arm HS-PEO/PNE coating on the Au sensor surface was conjugated with biotinylated-anti-HA protein aptamer. The aptamer binding buffer for conjugating biotin-aptamer to deglycosylated native avidin protein-maleimide/four arm HS-PEO/PNE was composed of 20 mM HEPES ((4-(2-hydroxyethyl)-1-piperazineethanesulfonic acid), 120 mM NaCl, 1 mM MgCl₂, 1 mM CaCl₂, 5 mM KCl and at pH 7.35, as per the manufacturer's instructions. The experimental surface was treated with 0.25 μM biotinylated-aptamer and the control surface (deglycosylated native avidin protein-maleimide-four arm HS-PEO/PNE) with buffer only. The incubation temperature was 25° C. Aptamer and buffer only solutions were perfused at 0.1 ml/min for 2 hours prior to QCM-I measurement under static conditions.

The HA protein solution (Sino Biological—HA specific to H1N1, A/Puerto Rico/8/1934) at a concentration of 2.5 μg/ml was then perfused at 20 μl/min on the sensor surfaces up to 30 hours at 25° C. Periodic recording was done for this time period with QCM-I. The buffer in which the virus was incubated was aptamer binding buffer. The frequency and dissipation changes were plotted with Excel. The bound mass (hydrated) was calculated with a GUI, denoted as NBS-QCM analysis, that utilizes Voigt's equation (Tables 6,7).

TABLE 6
Conjugated Biotinylated-Aptamer to Deglycosylated Native
Avidin Protein-maleimide - Four Arm HS-PEO/PNE Surface

| | Bound | | Bound Mass To |
|---|---|---|---|
| | Mass on | Bound Mass | deglycosylated |
| | Experimental | on Control | native avidin protein |
| | Sensor4 | Sensor | maleimide-four arm |
| Experiment | (ng/cm²) | (ng/cm²) | PEO/PNE (ng/cm²) |
| 1 | 767.6 | 497.1 | 270.5 |
| 2 | 249.9 | 82.7 | 332.6 |
| 3 | 260.9 | 36.3 | 224.6 |
| Average | 426.1 | 150.2 +/− 306.2 | 275.9 +/− 54.2 |

TABLE 7
Conjugated Biotinylated-Aptamer to Deglycosylated Native
Avidin Protein maleimide - Four Arm HS-PEO/PNE Surface

| | | | Bound |
|---|---|---|---|
| | | | Molecules To |
| | Bound | | deglycosylated |
| | Molecule on | Bound | native avidin |
| | Experimental | Molecules on | maleimide - four |
| | Sensor | Control Sensor | arm PEO/PNE |
| Experiment | (molecules/cm²) | (molecules/cm²) | (molecules/cm²) |
| 1 | 1.88 × 10¹³ | 1.22 × 10¹³ | 6.62 × 10¹² |
| 2 | 6.12 × 10¹² | 2.02 × 10¹² | 8.14 × 10¹² |
| 3 | 6.39 × 10¹² | 8.89 × 10¹¹ | 5.50 × 10¹² |
| Average | (1.04 +/− | (3.68 +/− | (6.76 +/− |
| | 0.724) × 10¹² | 7.50) × 10¹² | 1.33) × 10¹² |

Given the average number of molecules of deglycosylated native avidin protein maleimide bound (8.250±1.127)×10¹² molecules/cm² to the deglycosylated native avidin protein maleimide/four arm HS-PEO/PNE surface (Table 4), the bound molecule density of biotinylated aptamer (6.76±1.33)×10¹²/cm²) is close to the range expected for one biotinylated aptamer per one deglycosylated native avidin protein maleimide molecule.

### Example 6—Bonding of Biotinylated Aptamer to Deactivated H1N1 Virus in Suspension

Dynamic light scattering (DLS) was used to assess the binding of biotinylated aptamer to inactivated H1N1 virus (live virus obtained from ATCC-handled in BSL-2 facility in live form). The main parameter assessed was the diameter of the viral particles and how it increased when the biotinylated aptamer was incubated with inactivated virus. Inactivated viruses were generated by incubating live virus in 0.2% formalin for 18 hours. Inactivated viruses were suspended in aptamer binding buffer, which is composed of pH 7.35, 20 mM HEPES, 120 mM NaCl, 1 mM MgCl₂, 1 mM CaCl₂, 5 mM KCl, at a concentration of 1×10⁷ viral particles/ml. Biotinylated anti-HA protein aptamer was added to inactivated virus solutions to achieve a final concentration of 92 nM or 9.2 nM. A control condition in which no aptamer was added was also included for comparison. Inactivated viruses were incubated overnight for about 18 hours at 4° C. The next day, the three different conditions were assessed with DLS and showed that the HA on the inactivated virus bound to the supplied biotinylated aptamer in suspension in a concentration-dependent manner (Table 8).

TABLE 8
Dynamic Light Scattering (DLS) Assessment of The
Size of Inactivated H1N1 Conjugated with Aptamer

| | Virus Size with | Virus Size With | Virus Size With |
|---|---|---|---|
| | No Aptamer | 92 nM Aptamer | 9.2 nM Aptamer |
| Sample # | Treatment | Treatment (nm) | Treatment (nm) |
| 1 | 116 | 234 | 146 |
| 2 | 106.7 | 184 | 155 |
| 3 | 95.2 | 193 | 136 |
| Average | 106 +/− 10.4 | 207 +/− 23.2 | 146 +/− 9.56 |

### Example 7—Live H1N1 Virus Detection with QCM-I

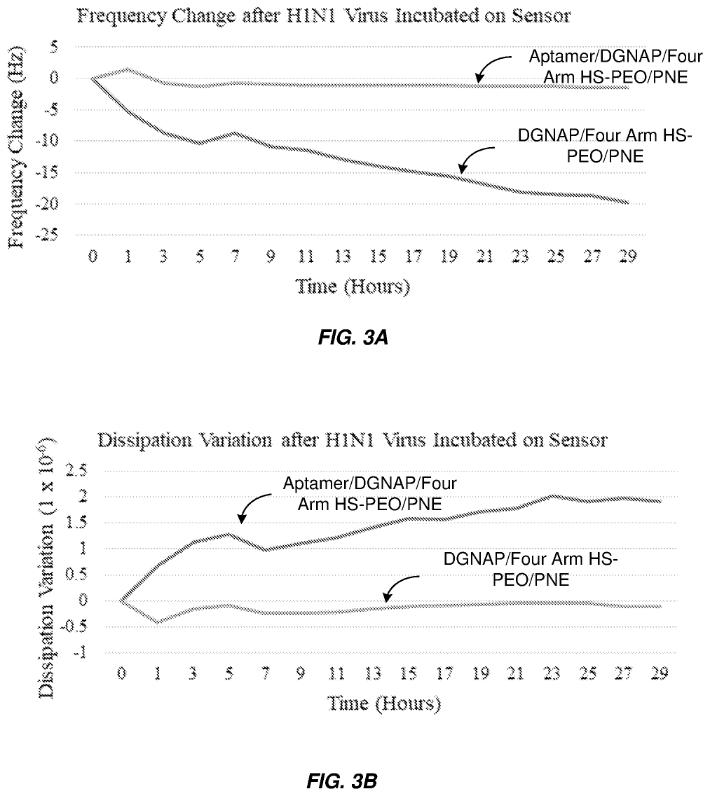

The experimental sensor surface (Aptamer/Deglycosylated native avidin protein (DGNAP) maleimide/four arm HS-PEO/PNE/Au) was compared to a control sensor surface (Deglycosylated native avidin protein (DGNAP) maleimide/four arm HS-PEO/PNE/Au). Live H1N1 solutions at a concentration of 1.5×10¹⁴ viral copies/ml were statically incubated on the sensor surfaces up to 29 hours in aptamer binding buffer at 25° C. while recording the frequency change and impedance over this time period on the QCM-I under fully hydrated conditions. FIG. 3A shows the frequency change and FIG. 3B shows the dissipation variation after static incubation of such viral sample (1.5×10¹⁴ viral copies/ml at 25° C. and n=1 for both conditions).

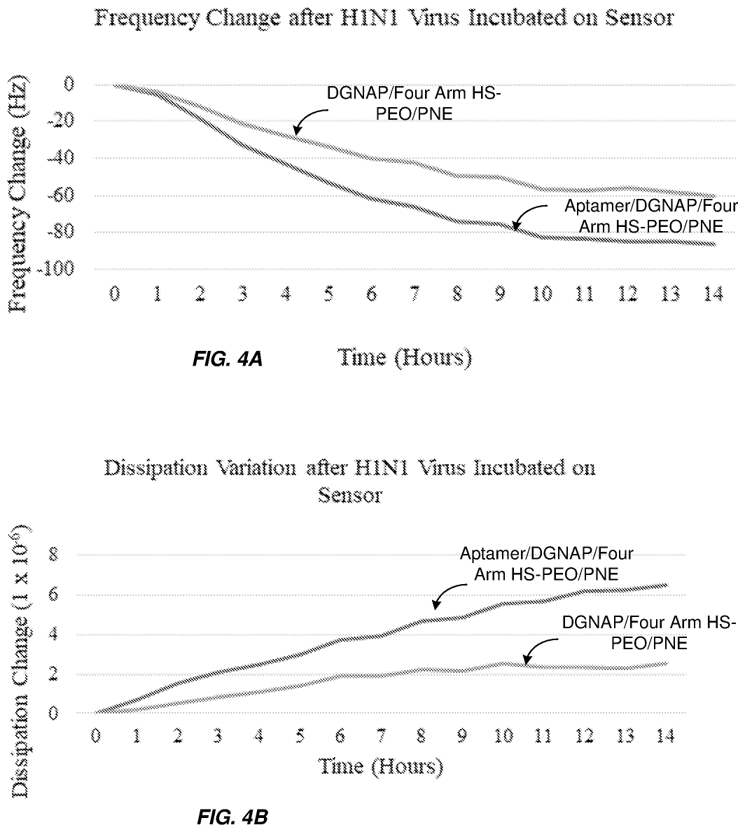

FIGS. 4A and 4B show viral detection under continuous convention conditions. Namely, FIG. 4A shows the frequency change and FIG. 4B shows the dissipation variation after 20 μl continuous perfusion of a live H1N1 viral sample (1×10¹³ viral copies/ml) for 14 hours at 25° C., n=1 for aptamer coated (experimental) and uncoated (control) conditions. The experimental sensor surface was functionalized with biotinylated aptamer on an Aptamer/Deglycosylated native avidin protein (DGNAP) maleimide/four arm HS-PEO/PNE surface. The control sensor surface was a deglycosylated native avidin protein (DGNAP) maleimide/four arm HS-PEO/PNE surface. Continuous recording was done for this time period on the QCM-I under fully hydrated conditions. The buffer in which the virus was incubated was aptamer binding buffer.

Continuous convection flow increased the amount of bound virus from 160 ng/cm² to 250 ng/cm², after the non-specific binding was subtracted. This increase also occurred in 14 hours, which is half the amount of time in which diffusion limited static measurement reached 160 ng/cm². In addition, the virus concentration used in the continuous perfusion experiment (1×10¹³ viral copies/ml) was lower than the concentration used in diffusion limited, static experiment (1×10¹⁴ viral copies/ml).

1. A method for functionalizing a sensor surface to selectively bind a target molecule comprising:
a. providing a sensor surface;
b. coating said surface with either polydopamine or polynorepinephrine;
c. providing a multi-arm poly(ethylene oxide) having the following formulae: 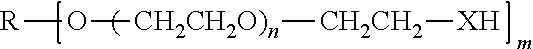
wherein n has a value of 10 to 45, m has a value of 2-12, R is an alkyl group providing carbon functionality to bond to the polyethylene oxide, X is S, —NH or O thereby providing available thiol, amine or hydroxy functionality;
d. covalently bonding a portion of said available thiol, amine or hydroxy functionality on said multi-arm poly(ethylene oxide) to said polydopamine or polynorepinephrine;
e. providing a maleimide conjugated deglycosylated native avidin protein (DGNAP) having biotin binding sites, said maleimide conjugated deglycosylated native avidin protein having the following general structure: 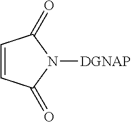
f. covalently bonding a portion of said available thiol, amine or hydroxy functionality on said multi-arm poly(ethylene oxide) to said maleimide conjugated deglycosylated native avidin protein having biotin binding sites.

2. The method of claim 1 wherein the value of m of said multi-arm poly(ethylene oxide) is in the range of 2-8.

3. The method of claim 1 wherein the value of m of said multi-arm poly(ethylene oxide) is in the range of 3-6.

4. The method of claim 1 wherein the value of n in said multi-arm poly(ethylene oxide) is in the range of 10-45.

5. The method of claim 1 wherein said coating of polydopamine or polynorepinephrine has a thickness in the range of 0.1 nm to 100 nm.

6. The method of claim 1 wherein said multi-arm poly(ethylene oxide) comprises the following structure:

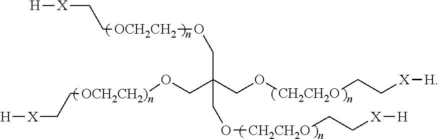

7. The method of claim 1 wherein said substrate surface comprises a quartz crystal microbalance sensor.

8. The method of claim 1 further including binding a biotinylated bait aptamer to said biotin binding sites, wherein said biotinylated bait aptamer binds to a specific target molecule and exposing said sensor surface to a fluid sample containing said targeted molecule and detecting the presence of said targeted molecule.

9. The method of claim 8 wherein said targeted molecule is associated with a target virus.

10. The method of claim 9 wherein said target virus is the H1N1 virus.

11. A method of detecting a targeted virus comprising:
a. providing a sensor surface coated with polydopamine or polynorepinephrine, covalently bonded to a multi-arm poly(ethylene oxide) wherein said multi-arm poly(ethylene oxide) is covalently bonded to a maleimide conjugated deglycosylated native avidin protein having biotin bonding sites including a bait biotinylated aptamer bonded to said biotin bonding sites;
b. exposing said sensor surface to a fluid sample containing a target virus and detecting the presence of said target virus.

12. The method of 10 wherein said polydopamine or polynorepinephrine coating has a thickness in the range of 0.1 nm to 100 nm.

13. The method of claim 10 wherein said sensor comprises a quartz crystal microbalance sensor.

14. The method of claim 10 wherein said target virus is the H1N1 virus.

15. A sensor for detecting a target molecule comprising a sensor surface coated with polydopamine or polynorepinephrine, covalently bonded to a multi-arm poly(ethylene oxide) wherein said multi-arm poly(ethylene oxide) is covalently bonded to a maleimide conjugated deglycosylated native avidin protein having biotin bonding sites which biotin bonding sites are bonded to a biotinylated bait aptamer wherein the biotinylated bait aptamer binds to a target molecule.

16. The sensor of claim 14 wherein said poly(ethylene oxide) in said multi-arm poly(ethylene oxide) has a molecular weight of 500 to 2000.

17. The sensor of claim 14 wherein said polydopamine or polynorepinephrine is coated on said sensor at a thickness of 0.1 nm to 100 nm.

18. The sensor of claim 14 wherein said sensor comprises a quartz crystal microbalance sensor.
